(19) United States
(12) Patent Application Publication
(10) Pub. No.: US 2014/0142683 A1
(43) Pub. Date: May 22, 2014

(54) STENT WITH ELASTOMERIC ELEMENTS

(71) Applicants: Lee Core, Needham, MA (US); Danny Concagh, Medfield, MA (US); Emily Rusk, Boston, MA (US); Stephanie Webber, Brookline, MA (US); Raymond Knox, Worcester, MA (US); Kircherl Ho, Groton, MA (US)

(72) Inventors: Lee Core, Needham, MA (US); Danny Concagh, Medfield, MA (US); Emily Rusk, Boston, MA (US); Stephanie Webber, Brookline, MA (US); Raymond Knox, Worcester, MA (US); Kircherl Ho, Groton, MA (US)

(21) Appl. No.: 14/086,070

(22) Filed: Nov. 21, 2013

Related U.S. Application Data
(60) Provisional application No. 61/728,873, filed on Nov. 21, 2012.

(51) Int. Cl. A61F 2/915

(52) U.S. Cl. CPC A61F 2/915
USPC 623/1.15; 29/515

(57) ABSTRACT

Disclosed is a self-expanding medical implant for placement within a lumen of a patient. The implant comprises a woven or non-woven structure having a substantially tubular configuration, and is designed to be low-profile such that it is deliverable with a small diameter catheter. The implant has a high recoverability and desirable mechanical properties.

### CROSS-REFERENCE TO RELATED APPLICATIONS

This application claims priority to and the benefit of, and incorporates herein by reference in its entirety, U.S. Provisional Patent Application No. 61/728,873, which was filed on Nov. 21, 2012.

### FIELD OF THE INVENTION

In various embodiments, the present invention relates generally to implantable stents and, more specifically, to stents that include elastomeric elements for providing enhanced stent expandability and/or mechanical properties.

### BACKGROUND

A variety of medical conditions are treatable by the implantation of tubular devices into natural body lumens or cavities. For example, it is commonplace to implant metallic stents into the coronary arteries of patients with heart disease following balloon angioplasty to minimize the risk that the arteries will undergo restenosis in the future. Generally, the implantable stents are forcibly compacted to a small diameter by a constraining sleeve or other means during delivery. Following delivery to the desired site, the stent constraints are released and the stent opens to contact the luminal surface of the body.

Recent developments in the field of implantable stents include the use of a tubular covering fitted to the stent, either to the outer surface, the luminal surface or to both surfaces of the stent to decrease the risk of restenosis. These covered stents have generally come to be referred to as stent-grafts. The coverings are generally of a polymeric biocompatible material such as polyethylene terephthalate (PET) or polytetrafluoroethylene (PTFE). Other examples of conventional tubular medical implants include woven grafts that are used to span vascular aneurysms, polymeric tubes and catheters that are used to bypass strictures in the ureter and urethra, and stents that are used in the peripheral vasculature, prostate, and esophagus.

Additionally, several approaches have been used to expand the diameter of a stent after delivery. For example, some stents require the use of a separate expansion device to mechanically apply a radially outward force on the stent walls to increase the stent diameter after delivery. Other stents are designed to be self-expanding, without the need for an extra mechanical expansion device. For example, the stents can be made from coiled or patterned nitinol metal alloys that are superelastic, biocompatible, and have shape memory. The stents are initially fashioned in an expanded state. Prior to implantation, the stent is tightly wound or crimped in order to reduce its diameter, Upon heating above a transition temperature, the shape memory alloy coil reverts to its original shape, with a larger diameter. Conventional self-expanding stents, however, may have limited expandability and mechanical properties.

Conventional stents may expand from their contracted states to their expanded states in a relatively arbitrary manner, e.g., at an inconsistent or variable rate and/or force. Physicians may have no control or selection over the expansion rate or force of such stents. Rapid expansion of a stent within a lumen such as a blood vessel may cause trauma to the lumen or to tissue surrounding the lumen, which may be undesirable. For example, smooth muscle areas lining a blood vessel in which a stent is rapidly expanding may tend to resist an abrupt enlargement of the blood vessel. The inability of the blood vessel walls to rapidly acclimate to the rapidly expanding diameter of the vessel may lead to damage to, or aggravate, the vessel wall. Such damage may lead to, or accelerate, re-occlusion or restenosis of the blood vessel. Restenosis of the vessel may require performance of an additional invasive intervention in the treated area within a relatively short period of time.

Consequently, there is a need for self-expanding stents that provide enhanced stent expandability and controlled expansion rates while being deployed in the desired target site.

### SUMMARY

In various embodiments, the present invention relates to an implantable tubular medical device for placement within a lumen or cavity of a patient. The device may be formed from a metallic material, such as stainless steel, or one or more polymer strands. In one implementation, the device includes a tubular shape that has multiple open areas and elements that span at least some of the open areas. The elements impart a force that urges proximal and distal portions of the open areas towards each other to thereby provide enhanced expandability and mechanical properties when compared with conventional self-expanding devices. Additionally, the current invention provides self-expanding devices with various controllable expansion forces and/or rates during self-expansion, thereby allowing the physician to select a device with a desirable expansion force and/or rate based on the properties of the target site.

Accordingly, in one aspect, the invention pertains to a medical implant. In various embodiments, the medical implant includes a tubular structure having a proximal end, a distal end, and a sidewall extending between the proximal and distal ends; the sidewall includes multiple open areas. In some embodiments, each open area includes one or more surfaces collectively defining a continuous outer boundary of the open area, a proximal boundary portion defined by the first portion of the surface(s), and a distal boundary portion defined by the second portion of the surface(s); the distal boundary portion is closer than the proximal boundary portion to the distal end of the tubular structure. In various embodiments, the medical implant further includes an element spanning one or more open areas between the proximal and distal boundary portions of the open area(s). In one implementation, when the tubular structure is in a reduced diameter configuration, a distance between the proximal and distal boundary portions is a first distance, and when the tubular structure is in an expanded diameter configuration, a distance between the proximal and distal boundary portions is a second distance; the first distance is greater than said second distance.

In another aspect, the invention relates to a method of manufacturing a medical implant as described herein.

In another aspect, the present invention relates to a method of treating a patient by implanting a medical implant as described herein into a blood vessel or other bodily lumen or cavity of the patient.

In another aspect, the present invention relates to a kit that includes a medical implant as described herein along with a suitable delivery device.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIGS. 1A-1C are side views of an implantable braided medical device in accordance with an embodiment of the invention.

FIGS. 2A and 2B illustrate the device in a natural state and a low-profile state, respectively, in accordance with an embodiment of the invention.

FIG. 3 depicts elements that span certain open areas of the implant in accordance with an embodiment of the invention.

FIGS. 4A and 4B illustrate elements on a device in its natural configuration and reduced diameter configuration, respectively, in accordance with embodiments of the invention.

FIGS. 5A-5C depict various approaches for forming an element on devices in accordance with various embodiments of the invention.

FIG. 6 is a side view of an implantable unitary framework medical device in accordance with an embodiment of the invention.

### DETAILED DESCRIPTION

The present invention provides for self-expanding medical implants that have expansion characteristics and mechanical properties that render them suitable for a broad range of applications involving placement within bodily lumens or cavities. As used herein, “device,” “implant,” and “stent” are used synonymously to mean any scaffold, endoprosthesis or other tubular structures that may be implanted into the human body. Also as used herein, “self-expanding” is intended to include devices that are crimped to a reduced configuration for delivery into a bodily lumen or cavity, and thereafter tend to expand to a larger suitable configuration once released from the delivery configuration, either without the aid of any additional expansion devices or with the partial aid of balloon-assisted or similarly-assisted expansion. When compared with conventional self-expanding medical implants, the implants of the present invention recover to a higher percentage of their manufactured diameter after being crimped and held in a small diameter for delivery into a bodily lumen. Moreover, when compared with conventional self-expanding implants and particularly polymeric implants, the implants of the present invention are characterized by much improved strength and other desired mechanical properties.

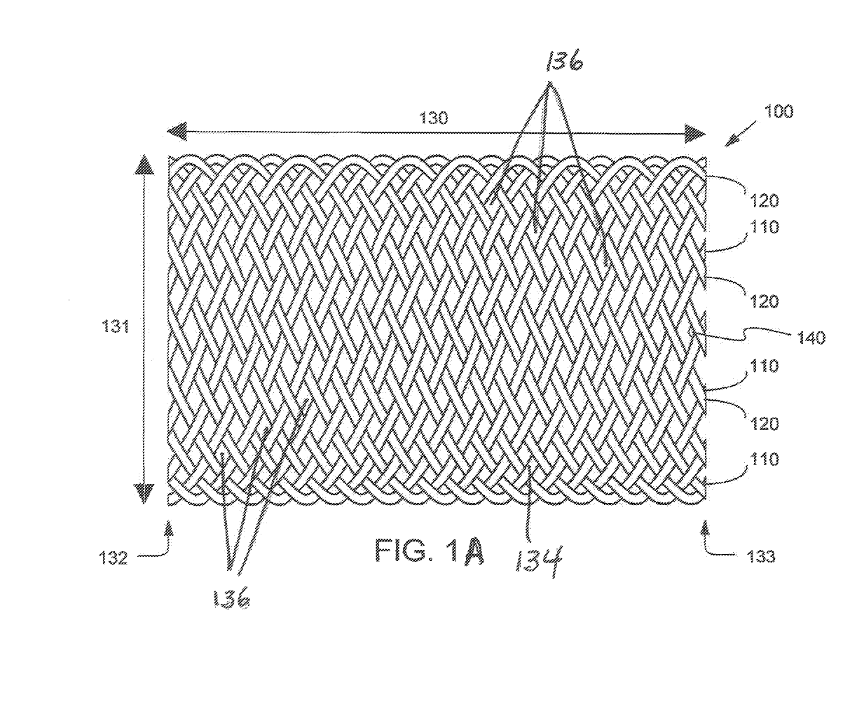

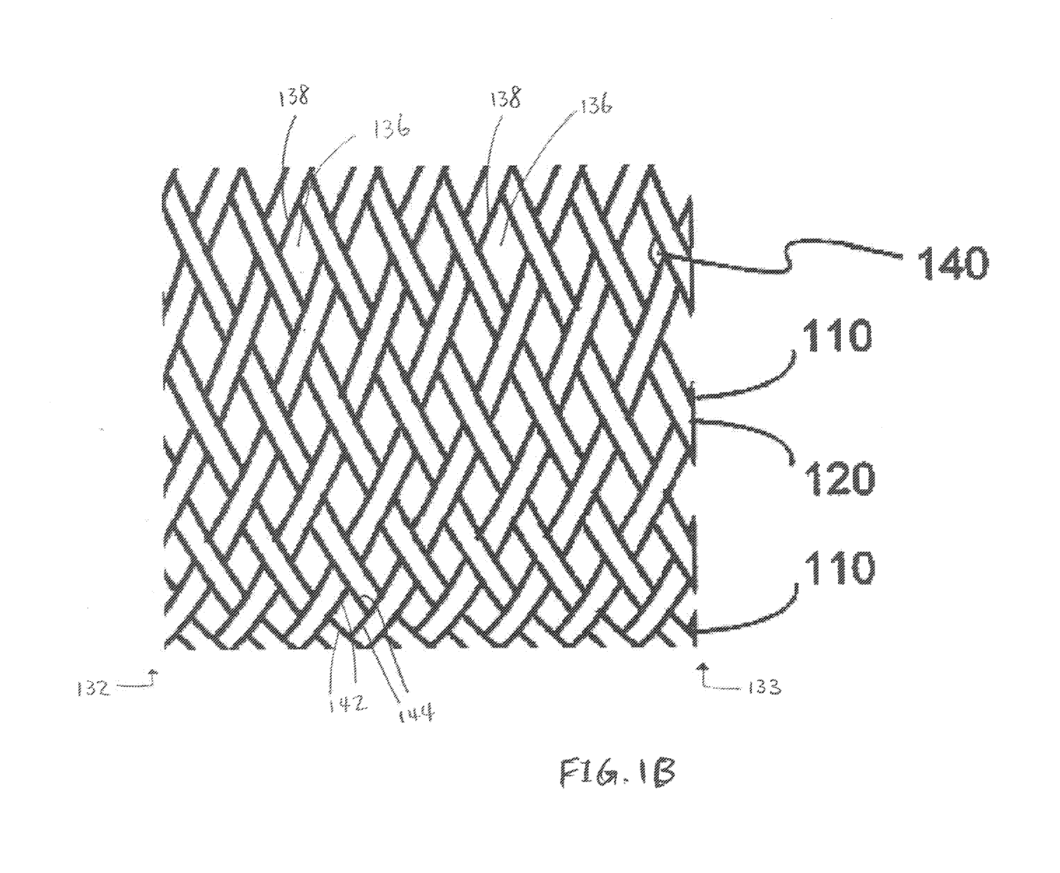

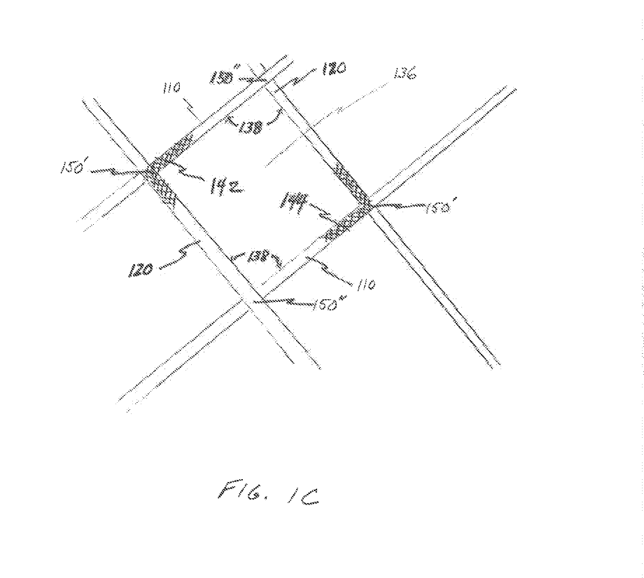

Examples of stent structures and related technology suitable for use with the present invention are described in U.S. Ser. No. 13/370,025, which is incorporated herein by reference for all purposes. In one embodiment shown in FIGS. 1A and 1B, the implant 100 preferably includes a substantially tubular configuration having a longitudinal dimension 130, a radial dimension 131, proximal and distal ends 132, 133 along the longitudinal dimension, and a sidewall 134 extending between the proximal and distal ends 132, 133. The tubular configuration, for example, may be woven using two sets of strands 110 and 120 to form the tubular sidewall 134 that has multiple open areas 136. As used herein, the term “strands” include fibers, extruded elements, struts, and other flexible and inflexible elements formed by any suitable method that are moveable with respect to each other. Also as used herein, “woven” is used synonymously with “braided.” Each set of the strands may extend in an opposed helix configuration along the longitudinal dimension of the implant 100. The sets of strands 110 and 120 may cross each other at a braid angle 140, which may be constant or may change along the longitudinal dimension of the implant. Preferably, there are between about 16 and about 96 strands used in the implants of the present invention, and the braid angle 140 is within the range of about 90 degrees to about 135 degrees throughout the implant. The strands are woven together using methods known in the art, using known weave patterns such as Regular pattern “1 wire, 2-over/2-under”, Diamond half load pattern “1 wire, 1-over/1-under”, or Diamond pattern “2 wire, 1-over/1-under”.

Referring to FIG. 1B, the strands 110, 120 may be in physical contact or sufficiently close to contact to each other to collectively form a surface 138 that defines a continuous outer boundary of each open area 136. The surface 138 includes proximal and distal boundary portions 142, 144. The proximal boundary portion 142 is closer to the proximal end 132 than the distal boundary portion 144 is; likewise, the distal boundary portion 144 is closer to the distal end 133 than the proximal boundary portion 142. An individual open area 136 is shown in FIG. 1C. Open area 136 is defined by the surface 138 formed by the strands 110, 120. Proximal and distal boundary portions 142, 144 are shown as shaded portions of the strands 110, 120 in FIG, IC. Generally, the proximal and distal portions include proximal and distal strand cross-over nodes 150′ and extend 10%, 20%, 30%, 40% or up to 50% of the distance between each proximal and distal strand cross-over node 150′ and its closest radial strand cross-over node 150″. Although the present invention is described with specific reference to woven structures, it should be appreciated that the invention is equally applicable to non-woven stents; embodiments of which are further described herein.

The strands 110, 120 may be made from biostable polymeric or metallic materials. Alternatively, they may be made from one or more biodegradable polymers or metals that are preferably absorbed within about two years of placement within a patient, and more preferably within about one year of placement within a patient. In some embodiments, the strands are fully absorbed within about six or fewer months of placement within a patient. The first and second strand sets 110, 120 may be made from the same or different biodegradable polymer. Non-limiting examples of biodegradable polymers that are useful in the strands of the present invention include poly lactic acid (PLA), poly glycolic acid (PGA), poly trimethylene carbonate (PTMC), poly caprolactone (PCL), poly dioxanone (PDO), and copolymers thereof. Preferred polymers are poly(lactic acid co-glycolic acid) (PLGA) having a weight percentage of up to about 24% lactic acid, or greater than about 79% lactic acid (preferably PLGA 88:12), with the former being stronger but degrading in the body faster. The composition of PLGA polymers within these ranges may be optimized to meet the mechanical property and degradation requirements of the specific application for which the implant is used. The term “biodegradable” is used herein synonymously with “bioabsorbable,” “bioerodible,” “resorbable,” and “bioresorbable” to describe a material or structure that degrades in the human body by any suitable mechanism.

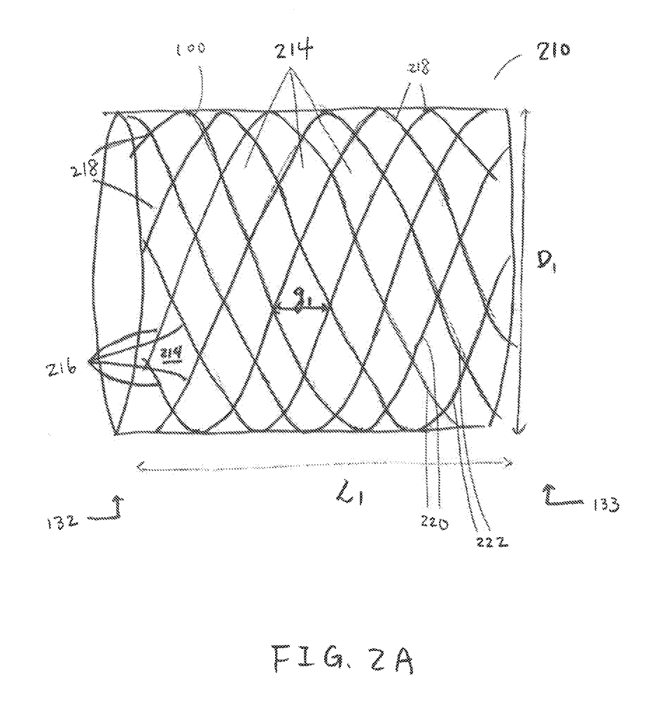

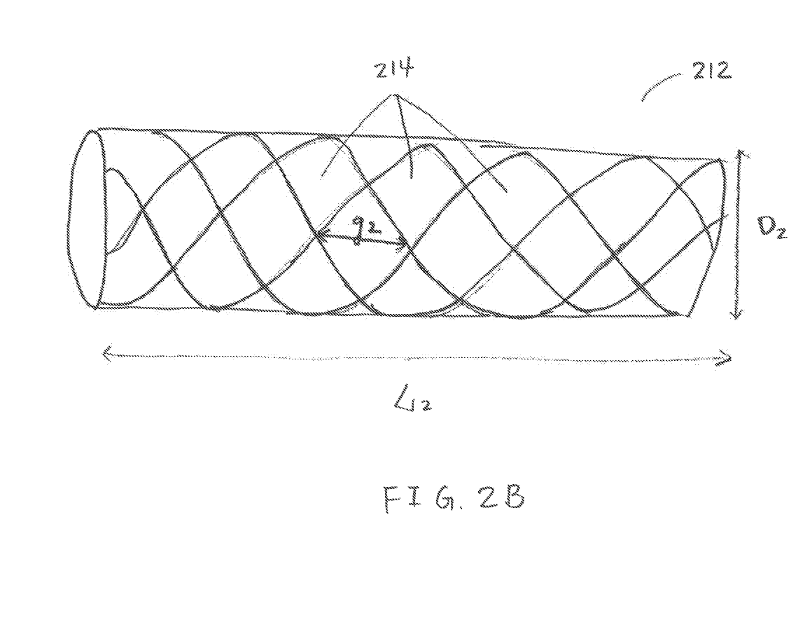

FIGS. 2A and 2B illustrate the implant 100 in a natural state 210 and a low-profile state (i.e., having a reduced diameter configuration) 212, respectively. As used herein, an implant is said to be in its “natural” state when it is in its unstressed, as-manufactured configuration. Referring to FIG. 2A, prior to delivery of the implant 100 into a small diameter bodily lumen or cavity, the implant 100 is in its natural state 210 with a diameter D₁ and an entire length L₁. As shown in the side view of FIG. 2A, the implant 100 is characterized by a plurality of open areas 214, each of which is defined by a continuous outer boundary 216 comprising one or more strands 218. The open areas 214 are said to be defined by the continuous outer boundary 216 because they appear to have such a boundary when the implant is viewed from the side, even if the boundary comprises strands that do not actually contact each other. The outer boundary 216 includes proximal and distal boundary portions 220, 222. The proximal boundary portion 220 is closer to the proximal end of the implant than the distal boundary portion 222; likewise, the distal boundary portion 222 is closer to the distal end than the proximal boundary portion 220. It should be noted that the terms “distal” and “proximal,” as used herein, are intended to refer to the blood flow inlet (proximal) and blood flow outlet (distal) of the implant. The manufactured diameter D₁ is preferably at least 10% larger than the diameter of the bodily lumen into which it is implanted. During delivery, referring to FIG. 2B, the implant 100 may be configured via crimped or other suitable mechanism into a low-profile state with a reduced diameter D₂ and an elongated length L₂ (i.e., D₂<D₁; L₂>L₁). As a result, the greatest distance between the proximal and distal boundary portions 220, 222 of each open area 214 is larger in the low-profile configuration than that in the unstressed, as manufactured configuration (i.e., g₂>g₁). The open areas 214 may be characterized by, for example, a diamond shape, an ovular shape, or any irregular shape formed by the strands 110, 120. As used herein, the term “irregular shape” is intended to include any shape that is not a regular geometric shape. Once the implant 100 is delivered to the target site, it may be released from the low-profile state, thereby self-expanding to a configuration having a larger diameter D₃>D₂; D₃ is preferable to be closer to D₁.

To facilitate the low-profile aspects of the present invention (e.g. during the delivery of the implants into small diameter bodily lumens or cavities), the strands used in the implant 100 preferably have a diameter in the range of from about 75 microns to about 500 microns, and are more preferably less than about 150 microns in diameter. The use of small diameter strands results in an implant with minimal wall thickness and the preferred ability to collapse (i.e., to be crimped) within low diameter catheter delivery systems. Where multiple strands are used, they may be of substantially equal diameters within this range, or first strand set 110 may be of a different general diameter than second strand set 120. In either event, the diameters of strands are chosen so as to render the implant 100 preferably deliverable from a 10 French delivery catheter (i.e., 3.3 mm diameter) or smaller, and more preferably from a 7 French delivery catheter. (i.e., 2.3 mm diameter) or smaller. The ability to place the implant of the present invention into small diameter delivery catheters allows for its implantation into small diameter bodily lumens and cavities, such as those found in the vascular, biliary, uro-genital, iliac, and tracheal-bronchial anatomy. Exemplary vascular applications include coronary as well as peripheral vascular placement, such as in the superficial femoral artery (SFA). It should be appreciated, however, that the implants of the present invention are equally applicable to implantation into larger bodily lumens, such as those found in the gastrointestinal tract.

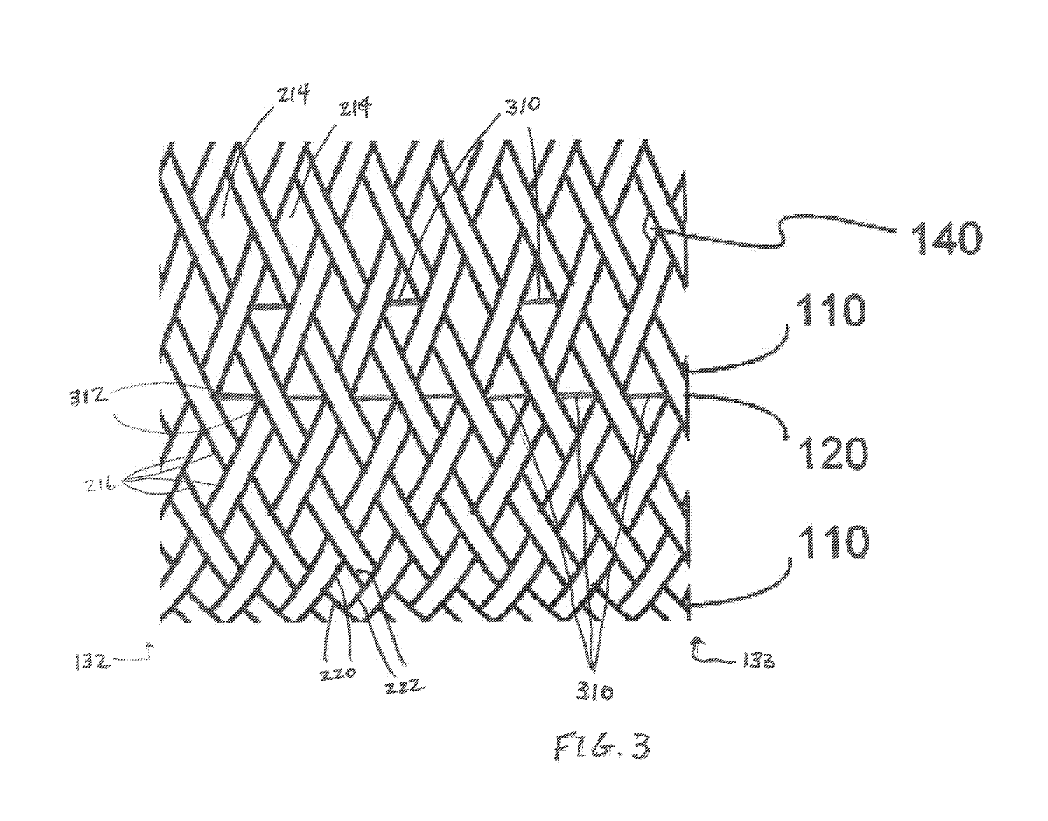

Referring to FIG. 3, in various embodiments, the implant comprises one or more elements 310 spanning one or more open area 214 of the tubular implant. The elements 310 preferably extend in a direction that is substantially parallel to the longitudinal dimension 130 of the tubular implant and span between the proximal boundary portion 220 and the distal boundary portion 222 of the open areas 214. For example, an element 310 may be placed across the space between two braid junctions 312 in the longitudinal dimension 130. As a result, the element 310 may connect strands within the same strand set or between different strand sets. In preferred embodiments, the elements 310 occupy a space less than the entire space within each open area. Elements 310 may be employed in every open area, every other open area, in a single area, or in any pattern or random placement throughout the implant sidewall.

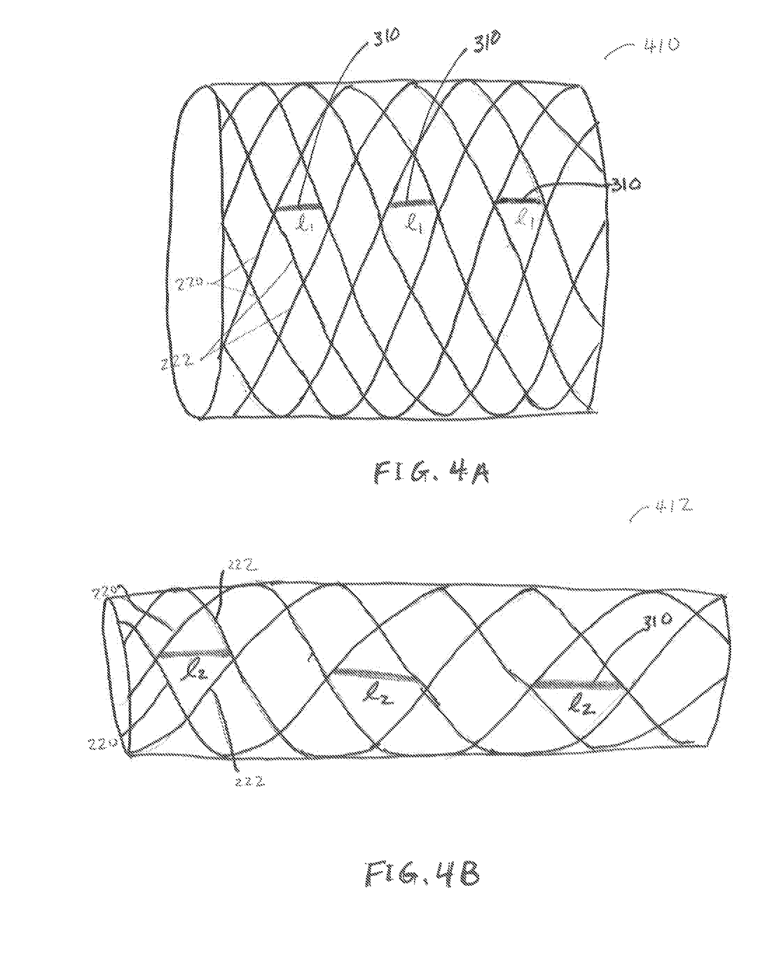

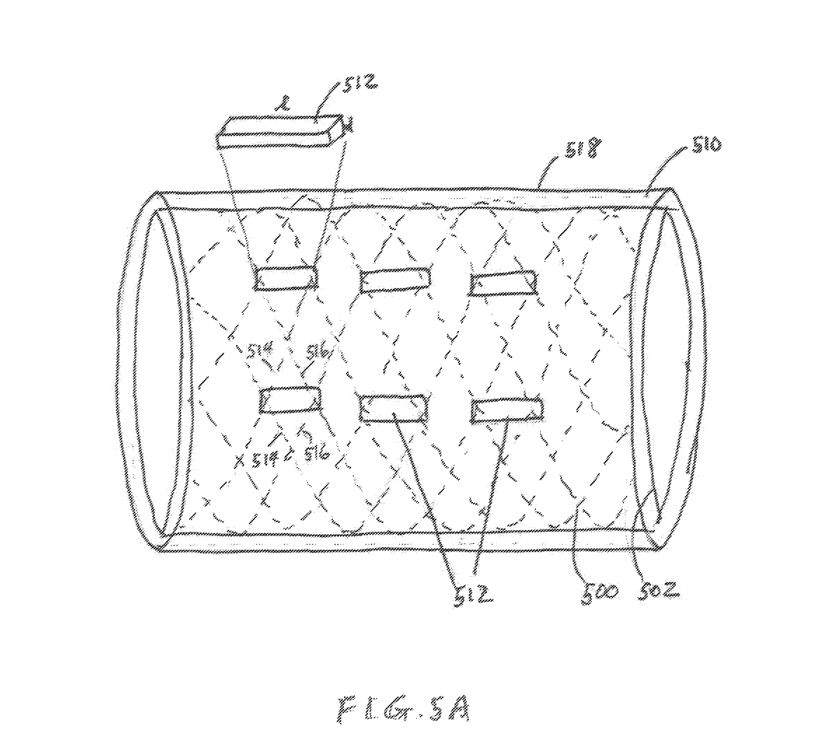

Referring to FIG. 4A, in some embodiments, the element 310 is formed when the implant is in its unstressed, as-manufactured state 410 and has a length l₁. Referring to FIG. 4B, when the implant is in the reduced diameter configuration 412, the element 310 is stretched and has a length l₂ (l₂>/l₁), which then imparts a tensile force (e.g., 0.2 N/cm² or more) to the proximal boundary portion 220 and the distal boundary portion 222 to which the element 310 is connected. Therefore, when the implant is released from the crimped delivery configuration, the stretched element 310 may contract to its equilibrium state of formation. This contraction force urges the proximal boundary portion 220 and the distal boundary portion 222 towards each other, thereby enhancing the expansion force and/or rate of the implant. Because the configuration of the implant may be easily varied before the element 310 is formed thereon, the stress on the element 310 resulting from the reduced diameter configuration of the implant crimp may be adjusted. As a result, the expansion force and/or rate of the implant following release from the delivery configuration may be tuned in a controlled way.

For example, the element 310 may be formed when the implant is in its unstressed, as-manufactured state (case I), 10% diameter reduced configuration (case II), or 50% diameter reduced configuration (case III). Upon placement of the stent into a reduced diameter configuration suitable for delivery into the body, the element 310 in case I is under the largest stress, whereas the element 310 in case III is under the smallest stress. When the implant is deployed at the desired target site, the element 310 in case I may create the largest contraction force, which then results in the highest expansion force and/or rate. By contrast, the element 310 in case III may generate the smallest contraction force, which may then result in the lowest expansion rate. Therefore, by adjusting the configuration (e.g., fully expanded, slightly crimped, or fully crimped) of the implant during the element formation, various degrees of stress on the strengthening means may be formed when the stents are in the low-profile state (e.g., having a reduced diameter configuration) during delivery. Once the stents are released from the low-profile state, the expansion force and/or rate of the implant upon being released from the delivery configuration may be well controlled and tuned (e.g., the stents that include a large stress strengthening means may expand with a higher expansion rate compared with those stents that include a small stress strengthening means). Accordingly, the current invention allows the physician to select an appropriate implant with a desirable expansion force and/or rate based on the tissue properties of the target site.

The element 310 may be made from an elastomeric polymer that, due to its elastic nature when compressed or elongated, applies a force to implant 100 that acts in favor of radial expansion and axial contraction, thus enhancing radial strength. The polymer of the connecting elements 310 is preferably biodegradable. Alternatively, the connecting elements 310 may be made from a shape memory material (e.g., nitinol) or a material that otherwise contracts upon heating to body temperature.

Examples of polymer materials used for the element 310 include suitable thermoplastic or thermoset elastomeric materials that yield the elongation, mechanical strength and low permanent deformation properties when combined with the implant strand(s). The inventors have found examples of suitable polymers to include certain random copolymers such as poly(lactic acid-co-caprolactone) (PLCL), poly(glycolide-co-caprolactone) (PGCL), and poly(lactic acid-co-dioxanone) (PLDO), certain homopolymers such as poly trimethylene carbonate (PTMC), and copolymers and terpolymers thereof. Such polymers are optionally crosslinked with a crosslinker that is bi- or multi-functional, polymeric, or small molecule to yield a thermoset polymer having a glass transition temperature (Tg) that is preferably lower than body temperature (37° C.), more preferably lower than room temperature (25° C.), and most preferably lower than about 5° C. The thermoset elastomers provide a high elongation to break with low permanent deformation under cyclic mechanical testing.

In one preferred embodiment, the polymer material used for the element 310 is a biodegradable thermoset elastomer synthesized from a four arm PGCL polymer having a weight ratio of approximately 50:50 GA:CL that is crosslinked with hexamethylene diisocyanate (HDI) to give a polyester with urethane crosslinks. Without wishing to be bound by theory, the inventors believe that the combination of the elastic segment (polyester portion) and the interactions (such as hydrogen bonding, allophanate or biuret formation) between the urethane segments of such polymers, in addition to a certain crosslinking density, yields preferred properties such as a high degree of elastic recovery under cyclic mechanical strain and high overall elasticity.

In other preferred embodiments, the element 310 comprises PLCL having about 45 to 75 weight percent lactic acid (e.g., a weight ratio of approximately 50:50 PL:CL). In yet another preferred embodiment, the element 310 comprises a PLCL 50:50 crosslinked with hexamethylene diisocyanate and the braided implant comprises a PLGA 79:21.

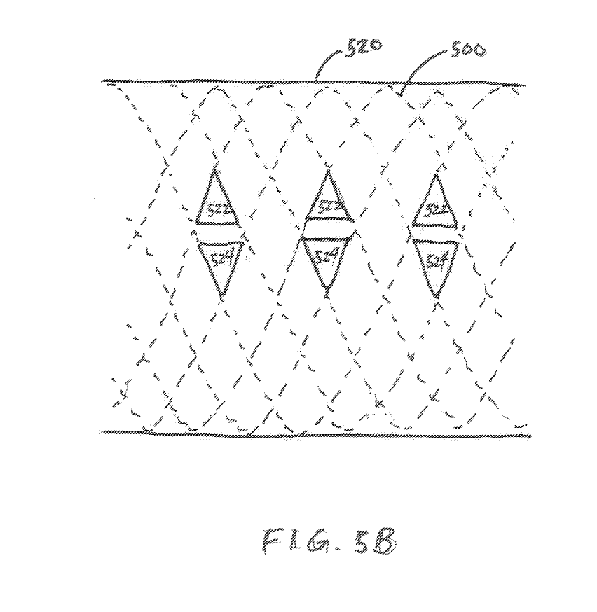

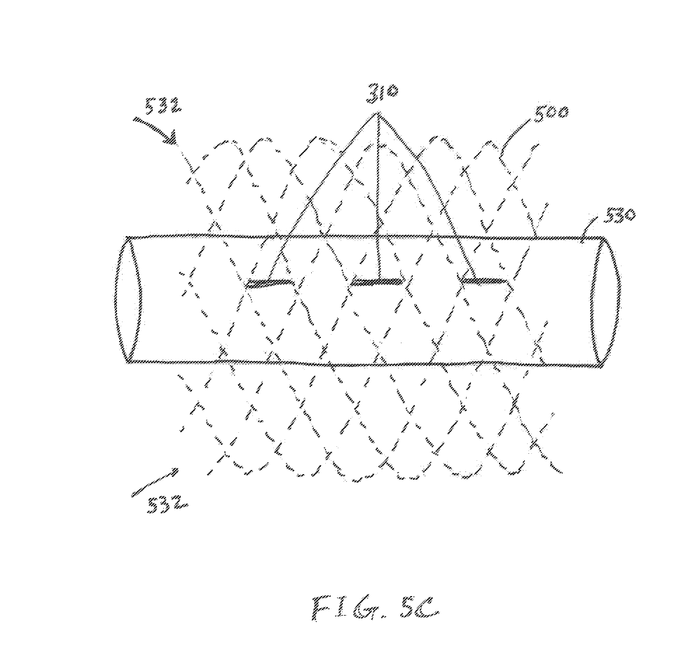

Methods for manufacturing exemplary tubular implants useful in the present invention are described in U.S. Ser. No. 13/370,025. Additionally, various techniques may be used to form the element 310 on the implant. Referring to FIG. 5A, for example, after the implant 500 is manufactured around a stainless steel mandrel 502, a circular mask 510 that has a diameter slightly larger than that of the implant may be used to cover the implant. The mask 510 includes one or more openings 512 that define areas where elements 310 may be formed. The openings 512 may be aligned with the strands on the implant such that the openings 512 span the space between the proximal boundary strand portion 514 and the distal boundary strand portion 516. The length l of the openings 512 is preferably long enough so that each opening 512 overlaps at least a portion of the proximal and distal strands; this helps ensure that the elements 310 will better attach to the strands. The depth d of the openings 512 determines the thickness of the elements 310. A film 518, made of an element forming material as described above, may be wrapped around the circular mask 510. The entire assembly is then placed in a suitable convection oven to allow the film 518 to melt, flow, and cure into the openings 512 where it attaches to the strands, thereby forming the elements 310. After which, the entire construction is removed from the oven and allowed to cool to approximately ambient temperature. The mask 510 is then carefully removed from the tubular implant; this leaves the elements 310 attached to the strands of the implant. Alternatively, the elements may be formed by spraying, dipping, electrospraying, rolling, or printing the element materials onto the space defined by the openings 512 of the mask 510.

Referring to FIG. 5B, in some embodiments, the manufactured tubular implant 500 is coated by a layer 520 of element forming materials; the coating methods are described in U.S. Ser. No. 13/370,025. The areas 522, 524 outside the predetermined space of the element can then be removed using any suitable means, including, for example, a scalpel blade, water jet, laser, etc. Additionally, any approaches and techniques that are suitable for forming the element on the strands of the tubular implant may be used and thereby within the scope of the current invention.

Referring to FIG. 5C, to form the element 310 on a reduced-diameter implant, a steel mandrel 530 having a diameter slightly less than the reduced implant diameter may be inserted into the manufactured implant. A force 532 may then be applied to the implant such that the implant 500 is crimped; the diameter of the implant is reduced to contact the mandrel 530. The approaches as described above may then be used to form the element on the implant. Because the diameter of the steel mandrel 530 may be easily changed or selected from a set of mandrels, the amount of the implant crimp may be adjusted when forming the element. This then allows the element to be stretched in various degrees during implant delivery, which in turn provides a tunable expansion force and/or rate during self-expansion upon deploying the implant.

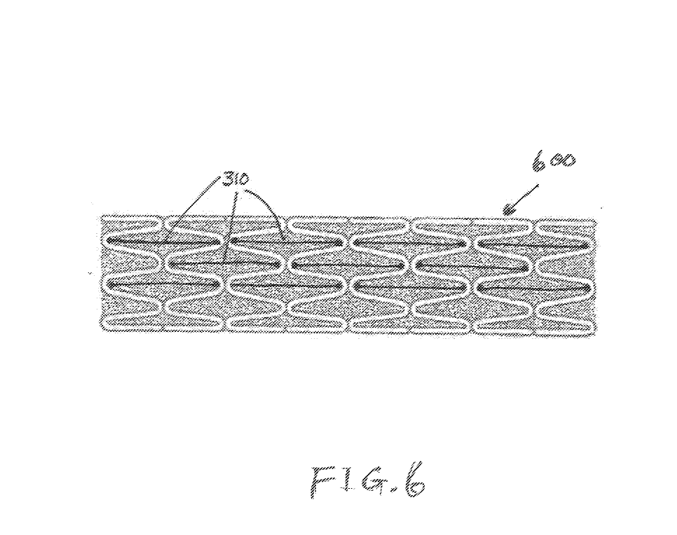

In another embodiment of the present invention, the implant is a non-woven, self-expanding structure, such as a unitary polymeric framework. As shown in FIG. 6, the non-woven implant 600 is preferably characterized by a regular, repeating pattern such as a lattice structure. The use of a unitary framework may provide a reduced profile when compared to the use of woven strands, which yield a minimum profile that is the sum of the widths of overlapping strands. In addition, a unitary framework eliminates the possible change in length of the implant associated with crimping and subsequent expansion, known as foreshortening, which is common in braided stents. When the implant 600 is a unitary framework, it is fabricated using any suitable technique, such as by laser cutting a pattern into a solid polymer tube. In a preferred embodiment, when the implant 600 is a unitary framework, it is formed by laser cutting and includes a wall thickness of between about 75 and about 150 microns. The implant includes elements 310, as previously described for woven stents. It should be recognized that while the present invention is described primarily with reference to woven strand configurations, aspects of the present invention are equally applicable to non-woven, self-expanding structures unless necessarily or expressly limited to woven configurations.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, routines, steps, or characteristics may be combined in any suitable manner in one or more examples of the technology. The terms “substantially” and “approximately” mean ±10% and, in some embodiments, within ±5%. The headings provided herein are for convenience only and are not intended to limit or interpret the scope or meaning of the claimed technology.

What is claimed is:

1. A medical implant, comprising:
a tubular structure having a proximal end, a distal end, and a sidewall extending between said proximal and distal ends, said sidewall comprising a plurality of open areas, each open area comprising:
at least one surface collectively defining a continuous outer boundary of said open area;
a proximal boundary portion defined by a first portion of said at least one surface; and
a distal boundary portion defined by a second portion of said at least one surface, the distal boundary portion being closer than the proximal boundary portion to the distal end of said tubular structure;
an element spanning at least one of said plurality of open areas between said proximal and distal boundary portions of said at least one of said plurality of open areas;
wherein when said tubular structure is in a reduced diameter configuration, a distance between said proximal and distal boundary portions is a first distance, and when said tubular structure is in an expanded diameter configuration, a distance between said proximal and distal boundary portions is a second distance, said first distance being greater than said second distance; and
wherein when said tubular structure is in said reduced diameter configuration, said element imparts a force to said proximal and distal boundary portions of the at least one of said plurality of open areas, said force acting to urge said proximal and distal boundary portions of the at least one of said plurality of open areas towards each other.

2. The medical implant of claim 1, wherein said element extends in a direction that is substantially parallel to a longitudinal axis of said tubular structure.

3. The medical implant of claim 1, wherein the tubular structure comprises at least one woven strand.

4. The medical implant of claim 3, wherein said strand is a polymeric material.

5. The medical implant of claim 4, wherein said polymeric material is a biodegradable material.

6. The medical implant of claim 1, wherein said tubular structure comprises metallic struts.

7. The medical implant of claim 6, wherein said metallic struts are biodegradable.

8. The medical implant of claim 1, wherein said element occupies less than the entire space within said open area.

9. The medical implant of claim 1, wherein said element comprises an elastomeric material.

10. The medical implant of claim 9, wherein said elastomeric material is selected from the group consisting of poly(lactic acid-co-caprolactone), poly(glycolide-co-caprolactone), poly trimethylene carbonate, and poly(lactic acid-co-dioxanone) and wherein said elastomeric material is cross-linked.

11. The medical implant of claim 9, wherein said elastomeric material comprises a random copolymer that is crosslinked.

12. The medical implant of claim 9, wherein said elastomeric material comprises poly(lactic acid-co-caprolactone) comprising about 45 to 75 weight percent lactic acid, wherein said polymer is cross-linked.

13. The medical implant of claim 12, wherein said poly(lactic acid-cocaprolactone) is crosslinked with hexamethylene diisocyanate.

14. The medical implant of claim 9, wherein the number average molecular weight (Mn) of said elastomeric material is greater than about 24,000 Da and less than about 100,000 Da.

15. The medical implant of claim 1, wherein said element comprises a shape memory material.

16. The medical implant of claim 15, wherein said shape memory material is nitinol.

17. The medical implant of claim 1, wherein said open area is characterized by a diamond shape when said tubular structure is in said reduced diameter configuration.

18. The medical implant of claim 1, wherein said open area is characterized by an ovular shape when said tubular structure is in said reduced diameter configuration.

19. The medical implant of claim 1, wherein said open area is characterized by an irregular shape when said tubular structure is in said reduced diameter configuration.

20. The medical implant of claim 1, wherein said force is at least 0.2 N/cm².

21. A method of manufacturing a medical implant, comprising:
providing a tubular structure having a proximal end, a distal end, and a sidewall extending between said proximal and distal ends, said sidewall comprising a plurality of open areas, each open area comprising:
at least one surface defining a continuous outer boundary of said open area;
a proximal boundary portion defined by a first portion of said at least one surface; and
a distal boundary portion defined by a second portion of said at least one surface, the distal boundary portion being closer than the proximal boundary portion to the distal end of said tubular structure; and
forming an element spanning at least one of said plurality of open areas, said element being in connection with each of said proximal and distal boundary portions of said at least one of said plurality of open areas,
wherein when said tubular structure is in a reduced diameter configuration, a distance between said proximal and distal boundary portions is a first distance, and when said tubular structure is in an expanded diameter configuration, a distance between said proximal and distal boundary portions is a second distance, said first distance being greater than said second distance; and
wherein when said tubular structure is in said reduced diameter configuration, said element imparts a force to said proximal and distal boundary portions of the at least one of said plurality of open areas, said force acting to urge said proximal and distal boundary portions of the at least one of said plurality of open areas towards each other.
